(19) United States
(12) Patent Application Publication
(10) Pub. No.: US 2007/0032551 A1
(43) Pub. Date: Feb. 8, 2007

(54) Salt of dimethylaminomethyl-phenyl-cyclohexane and crystalline forms thereof

(75) Inventors: Michael Gruss, Aachen (DE); Wolfgang Hell, Aachen (DE); Joerg Berghausen, Loerrach (DE); Martin Szelagiewicz, Muenchenstein (CH); Markus Von Raumer, Arlesheim (CH); Susan Margaret De Paul, Zurich (CH)

Correspondence Address: CROWELL & MORING LLP;INTELLECTUAL PROPERTY GROUP, P.O. BOX 14300, WASHINGTON, DC, 20044-4300, US

(73) Assignee: GRUENENTHAL GMBH, AACHEN (DE)

(21) Appl. No.: 11/491,100

(22) Filed: Jul. 24, 2006

Related U.S. Application Data
(60) Provisional application No. 60/702,641, filed on Jul. 27, 2005.

(30) Foreign Application Priority Data
Jul. 22, 2005 (DE) 10 2005 034 973.0

(51) Int. Cl. A61K 31/205

(52) U.S. Cl. 514/554; 562/595

(57) ABSTRACT

Novel 3-[2-(dimethylamino)methyl-(cyclohex-1-yl)]-phenol fumarate compounds corresponding to formula I: 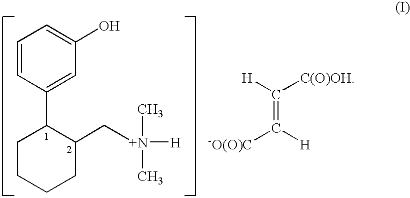 and processes for preparing these compounds are provided. Pharmaceutical compositions including these compounds and methods of treating or alleviating pain with these compounds are also provided.

### CROSS-REFERENCE TO RELATED APPLICATIONS

This application claims benefit under 35 U.S.C. § 119(e) to U.S. provisional patent application Ser. No. 60/702,641, filed Jul. 27, 2005, and also claims benefit to German patent application Serial No. 10 2005 034 973.0 filed July 22, 2005, the entire disclosures of which are hereby incorporated in their entirety.

### FIELD OF THE INVENTION

The present invention relates to a salt of fumaric acid and 3-[2-(dimethylamino)methyl-(cyclohex-1-yl)]-phenol, preferably in a composition of 1:1, stable crystalline forms of the salt and processes for the preparation thereof, a pharmaceutical composition and the use of the salt as a pharmaceutical active compound in a composition.

### BACKGROUND OF THE INVENTION

EP-A1-0 753 506 discloses 3-[2-(dimethylamino)methyl-(cyclohex-1-yl)]-phenols having an analgesic action. It is mentioned in the text that salts can also be prepared from the free bases, fumaric acid also being mentioned as a possible anion. In the examples, exclusively hydrochlorides are provided, that is to say a salt having a monovalent anion. EP-A1-0 753 506 contains no indications at all as to in what stoichiometry 3-[2-(dimethylamino)methyl-(cyclohex-1-yl)]-phenols having bivalent anions can exist, for example as hemi- or 1:1-salts. Closer investigation of 3-[2-(dimethylamino)methyl-(cyclohex-1-yl)]-phenol hydrochloride has shown that this crystalline solid substance is distinguished by a pronounced polymorphism and forms several crystalline, also metastable forms. In addition, this hydrochloride tends markedly to the formation of hydrates and solvates, which is a considerable disadvantage for targeted preparation of a specific crystalline form. Crystalline 3-[2-(dimethylamino)methyl-(cyclohex-1-yl)]-phenol hydrochloride has moreover proved to be decidedly hygroscopic. This properties profile of 3-[2-(dimethylamino)methyl-(cyclohex-1-yl)]-phenol hydrochloride illustrates that it is very difficult to provide pharmaceutical compositions having reproducible properties, which can also be retained over a storage period, with this active substance. To achieve these aims, expensive protective measures would at least be necessary.

### SUMMARY OF THE INVENTION

It has now been found, surprisingly, that with fumaric acid, 3-[2-(dimethylamino)methyl-(cyclohex-1-yl)]-phenol forms a fumarate as a crystalline solid substance, preferably in a composition in the ratio of 1:1 of fumarate and 3-[2-(dimethylamino)methyl-(cyclohex-1-yl)]-phenol. It has furthermore been found, surprisingly, that the fumarate is not hygroscopic, is stable in air and forms no hydrates or solvates. It has also been found, surprisingly, that 3-[2-(dimethylamino)methyl-(cyclohex-1-yl)]-phenol fumarate is capable of forming only few forms which are stable at a low or at a higher temperature, form B being the form which is stable at room temperature. Form A, which is stable at a higher temperature, can likewise be converted into form B, it being possible for the two forms to be in a mixture at a low temperature. The crystalline form B is also distinguished by a high chemical resistance at temperatures below 100° C. 3-[2-(Dimethylamino)methyl-(cyclohex-1-yl)]-phenol fumarate furthermore also has valuable biological properties, such as, for example, good solubility, particularly in polar and protic solvents, including water, and a good bioavailability. On the basis of its properties profile, 3-[2-(dimethylamino)methyl-(cyclohex-1-yl)]-phenol fumarate is very particularly suitable for formulation of pharmaceutical compositions.

### BRIEF DESCRIPTION OF THE DRAWINGS

FIG. 1 shows the X-ray diffraction diagram of polymorphic form B

FIG. 2 shows the Raman spectrum of polymorphic form B

FIG. 3 shows the X-ray diffraction diagram of polymorphic form A

FIG. 4 shows the Raman spectrum of polymorphic form A.

### DETAILED DESCRIPTION

The invention firstly provides salts of fumaric acid with 3-[2-(dimethylamino)methyl-(cyclohex-1-yl)]-phenol, 3-[2-(dimethylamino)methyl-(cyclohex-1-yl)]-phenol fumarate of the formula I 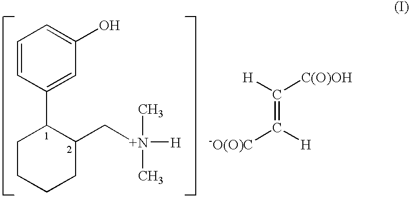
being preferred.

The compounds according to the invention such as the compounds of the formula I contain a chiral C atom in each of the 1 and 2 positions of the cyclohexane ring. The compounds according to the invention such as the compounds of the formula I include all the stereoisomers and mixtures of stereoisomers. Diastereomers or mixtures of enantiomeric diastereomers having the trans configuration of the phenyl ring and of the dimethylaminomethyl group (1R,2R or 1S,2S configuration) are preferred, the enantiomer having the absolute configuration (1R,2R) being very particularly preferred.

The structure of the (1R,2R) enantiomer of 3-[2-(dimethylamino)methyl-(cyclohex-1-yl)]-phenol is shown below:

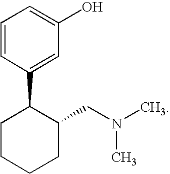

The compounds of the formula I can be obtained, analogously to the processes described generally in EP-A1-0 753 506 for the preparation of salts, from the free base by reaction with fumaric acid in the presence of water. The free base 3-[2-(dimethylamino)methyl-(cyclohex-1-yl)]-phenol can be isolated, for example, from the hydrochloride described in Examples 9 and 10 of EP-A1-0 753 506. For this, the hydrochloride is dissolved in an organic solvent, an aqueous inorganic base, for example alkali metal bases or also alkali metal bicarbonates (such as LiOH, NaOH, KOH, NaHCO₃ and KHCO₃), is added and the organic phase is separated off. The organic phase can be dried, and the base is either isolated in the conventional manner, or used directly for the salt formation, optionally after concentration by evaporation of the solvent.

The invention also provides a process for the preparation of 3-[2-(dimethylamino)methyl-(cyclohex-1-yl)]-phenol fumarate of the formula I, comprising combining

3-[2-(dimethylamino)methyl-(cyclohex-1-yl)]-phenol and fumaric acid, at least one of the components preferably being present in dissolved or suspended form.

The invention also provides a process for the preparation of 3-[2-(dimethylamino)methyl-(cyclohex-1-yl)]-phenol fumarate of the formula I comprising the steps
a) dissolving or suspending of 3-[2-(dimethylamino)methyl-(cyclohex-1-yl)]-phenol in a solvent, or initial introduction of 3-[2-(dimethylamino)methyl-(cyclohex-1-yl)]-phenol into the reaction vessel in undissolved form,
b) mixing of the solution, suspension or undissolved substance with fumaric acid or a suspension or a solution of fumaric acid in a solvent, optionally cooling and keeping the mixture at a low temperature, and
c) isolation of the compound of the formula I,
   the temperature preferably being above 90° C. in no process stage and it also being possible to interchange steps a) and b).

It has been found, surprisingly, that the salt formation leads to only one crystalline form, namely form B, if the temperature is controlled and no temperatures above 90° C., preferably above 60° C., and particularly preferably above 40° C. are used.

The invention therefore also provides a process for the preparation of 3-[2-(dimethylamino)methyl-(cyclohex-1-yl)]-phenol fumarate of the formula I in the crystalline form B, comprising the steps
a) dissolving or suspending of 3-[2-(dimethylamino)methyl-(cyclohex-1-yl)]-phenol in a solvent, or initial introduction of 3-[2-(dimethylamino)methyl-(cyclohex-1-yl)]-phenol into the reaction vessel in undissolved form,
b) mixing of the solution, suspension or undissolved substance with fumaric acid or a suspension or a solution of fumaric acid in a solvent, optionally cooling and keeping the mixture at a low temperature until formation of the crystalline form B is complete, and
c) isolation of the compound of the formula I in the crystalline form B,
   the temperature being above 90° C. in no process stage and it also being possible to interchange steps a) and b).

The temperature in process stage b) during the mixing is preferably not more than 70° C., more preferably not more than 60° C., and particularly preferably not more than 40° C. Cooling can mean a temperature down to about −20° C., more preferably down to −10° C., and particularly preferably down to 0° C. During the dissolving according to process stage a), the temperature is in general higher than during the mixing according to process stage b). If a temperature above 40° C. is used during the dissolving, the solution is cooled to 40° C. or below before the mixing, and is optionally kept at this temperature for some time.

The free base and fumaric acid can be employed in the molar ratio of 1:1, or fumaric acid can also be used in excess, for example in a molar ratio of up to 1.3, preferably up to 1.1. If an excess of the free base is used, no hemi-fumarates are formed, even if a molar ratio of base to fumaric acid of 2:1 is established.

The amount of 3-[2-(dimethylamino)methyl-(cyclohex-1-yl)]-phenol in the solution can be, for example, 5 to 70 wt. %, preferably 10 to 60 wt. %, more preferably 10 to 50 wt. %, and particularly preferably 15 to 40 wt. %, based on the solution. The solution of 3-[2-(dimethylamino)methyl-(cyclohex-1-yl)]-phenol can be heated and then optionally cooled to the temperature desired for the mixing with fumaric acid.

Inert (compatible) solvents for 3-[2-(dimethylamino)methyl-(cyclohex-1-yl)]-phenol and fumaric acid are, for example, aliphatic, cycloaliphatic and aromatic hydrocarbons (hexane, heptane, petroleum ether, cyclohexane, methylcyclohexane, benzene, toluene, xylene), aliphatic halohydrocarbons (methylene chloride, chloroform, di- and tetrachloroethane), nitriles (acetonitrile, propionitrile, benzonitrile), ethers (diethyl ether, dibutyl ether, t-butyl methyl ether, ethylene glycol dimethyl ether, ethylene glycol diethyl ether, diethylene glycol dimethyl ether, tetrahydrofuran, dioxane), ketones (acetone, 2-butanone, methyl isobutyl ketone), carboxylic acid esters and lactones (ethyl or methyl acetate, valerolactone), N-substituted lactams (N-methylpyrrolidone), carboxylic acid amides (dimethylacetamide, dimethylformamide), acyclic ureas (dimethylimidazoline), and sulfoxides and sulfones (dimethyl sulfoxide, dimethyl sulfone, tetramethylene sulfoxide, tetramethylene sulfone) and alcohols (methanol, ethanol, 1- or 2-propanol, n-, i- and t-butanol, 1-pentanol, 1-hexanol, 1-heptanol 1-octanol, ethylene glycol monomethyl ether, ethylene glycol monoethyl ether, diethylene glycol monomethyl or monoethyl ether) and water. The solvents can be used by themselves or in a mixture of at least two solvents. Physiologically acceptable solvents with which the person skilled in the art is familiar are advantageously used.

The mixing in process stage b) can be carried out by means of slow or rapid addition of one solution to the other solution. One or both solutions can be heated. However, the mixing can also be carried out by a procedure in which one or both solutions are at room temperature or are cooled, for example down to −20° C., and more preferably down to −10 to +10° C., extremely preferably −5 to +5° C. After the mixing, the mixture can be heated and cooled again, and can be further stirred for a certain period of time. Crystal formation can also be promoted by seeding.

As a rule, a white precipitate which is crystalline and can easily be filtered is already formed during the mixing. The isolation can be carried out by decanting, filtering or centrifuging. The crystalline residue can then also be dried, for example by means of heating, vacuum drying or heating in vacuo, or by means of an optionally heated and inert gas stream (air, nitrogen, noble gases). The compounds of the formula I are obtained in high yields and a high purity. As a rule, no or only few purification steps, such as, for example, recrystallization, are necessary, and the product can be used directly for the preparation of pharmaceutical compositions.

The present invention also provides a salt of fumaric acid and 3-[2-(dimethylamino)methyl-(cyclohex-1-yl)]-phenol, especially a salt of the formula I, obtainable by one of the processes described above.

The compounds of the formula I and the preparation process according to the invention offer considerable and in some cases unexpected advantages over corresponding hydrochlorides. Since it was not possible to characterize any hydrates and solvates, the choice of solvents which can be employed is wide and non-critical. The stability in air and to moisture allows open handling without particular protective measures. The spontaneous salt formation and formation of crystalline precipitates and the good filterability thereof allow processes on an industrial scale.

The compounds of the formula I are obtained in the process according to the invention of salt formation as a crystalline solid predominantly in a polymorphic form, which is called form B in the following. Amorphous forms of the compounds of the formula I are easily obtainable, for example by means of freeze drying or rapid cooling of solutions. Amorphous compounds of the formula I are not very stable and tend towards crystallization in the presence of moisture. At temperatures above 40° C., mixtures of the crystalline form B and the crystalline form A, which is thermodynamically more stable at higher temperatures, form. The amorphous form is particularly suitable as a starting material for the targeted preparation of crystalline forms.

It has been found that the compounds of the formula I, as crystalline solids, form polymorphic forms which can be prepared in a targeted manner from 3-[2-(dimethylamino)methyl-(cyclohex-1-yl)]-phenol fumarate, and because of their stability are particularly suitable as an active compound for formulation of pharmaceutical compositions. It is known [see, for example, Z. Jane Li et al. in J. Pharm. Sci., vol. 88(3), pages 337 to 346 (1999)] that enantiomers give identical X-ray diffractograms and Raman spectra, and thus form the same polymorphic forms. Polymorphic forms of all the enantiomers are thus included in the context of the invention.

The invention also provides a crystalline form B of 3-[2-(dimethylamino)methyl-(cyclohex-1-yl)]-phenol fumarate of the formula I which has a characteristic X-ray diffraction diagram with the pronounced reflections given below.

| | rel. |
|---|---|
| 2 theta | intensity |
| 9.52 | 100 |
| 10.40 | 12 |
| 12.61 | 21 |
| 13.28 | 34 |
| 16.55 | 72 |
| 17.05 | 24 |
| 17.23 | 8 |
| 19.14 | 80 |
| 19.60 | 33 |
| 20.86 | 94 |
| 21.27 | 33 |
| 21.95 | 11 |
| 22.50 | 27 |
| 23.51 | 27 |
| 23.87 | 7 |
| 24.96 | 14 |
| 25.42 | 32 |
| 25.72 | 14 |
| 26.76 | 35 |
| 27.07 | 12 |
| 28.03 | 5 |
| 28.87 | 7 |
| 29.26 | 18 |
| 30.04 | 4 |
| 30.36 | 6 |
| 30.74 | 5 |
| 31.63 | 13 |
| 32.76 | 5 |
| 33.47 | 5 |
| 34.86 | 10 |
| 35.16 | 5 |
| 36.15 | 6 |
| 37.82 | 5 |
| 38.88 | 6 |
| 39.76 | 9 |
| 41.57 | 7 |
| 42.44 | 5 |
| 44.44 | 5 |
| 45.83 | 5 |
| 46.67 | 4 |

The above Table shows the peak positions (in 2 theta) and the relative intensities of the peaks, the most intense peak being standardized to a relative intensity of 100.

The invention also provides a crystalline form of 3-[2-(dimethylamino)methyl-(cyclohex-1-yl)]-phenol fumarate of the formula I which has a characteristic X-ray diffraction diagram in the range of from 2° to 35° 2θ with pronounced characteristic lines, expressed in d values (Å):
9.3 (vs) 7.0 (m), 6.7 (s), 5.37 (s), 5.21 (s), 4.64 (s), 4.52 (s), 4.28 (vs), 4.23 (s), 4.19 (s), 3.94 (m), 3.78 (m), 3.52 (m), 3.49 (m), 3.33 (s), 3.30 (m), 3.06 (s), 2.83 (m);
   called form B in the following.

Above and in the following, the abbreviations in parentheses denote: (vs)=very strong intensity, (s)=strong intensity, (m)=moderate intensity, (w)=weak intensity, and (vw)=very weak intensity. The abbreviation “sh” in the Raman spectra tables denotes shoulder.

The invention also provides a crystalline form of 3-[2-(dimethylamino)methyl-(cyclohex-1-yl)]-phenol fumarate of the formula I which has a characteristic Raman spectrum with characteristic lines, expressed in wave numbers (cm⁻¹):

| Position (cm⁻¹) | Intensity |
|---|---|
| 3070 | m |
| 3051 | m |
| 3031 | m |
| 3016 | m |
| 2991 | w |
| 2964 | m |
| 2930 | s |
| 2918 | s |
| 2898 | sh, m |
| 2878 | m |
| 2859 | s |
| 2813 | vw |
| 1683 | m |
| 1601 | m |
| 1472 | w |
| 1459 | m |
| 1444 | m |
| 1388 | m |
| 1350 | w |
| 1331 | w |
| 1323 | w |
| 1307 | w |
| 1295 | m |
| 1267 | w |
| 1246 | w |
| 1240 | w |
| 1236 | w |
| 1211 | w |
| 1177 | w |
| 1162 | w |
| 1121 | vw |
| 1106 | w |
| 1083 | w |
| 1074 | w |
| 1058 | w |
| 1048 | w |
| 1000 | vs |
| 972 | w |
| 957 | m |
| 930 | w |
| 905 | w |
| 857 | w |
| 841 | m |
| 821 | w |
| 797 | w |
| 763 | vw |
| 753 | m |
| 704 | vw |
| 634 | w |
| 620 | vw |
| 610 | vw |
| 571 | vw |
| 535 | m |
| 512 | vw |
| 469 | vw |
| 453 | vw |
| 412 | w |
| 355 | w |
| 347 | w |
| 325 | w |
| 279 | m |
| 246 | s |
| 239 | sh, m |
| 171 | m |
| 109 | vs |
| 89 | vs |
| 78 | vs |

called form B in the following.

The invention also provides a crystalline form B of 3-[2-(dimethylamino)methyl-(cyclohex-1-yl)]-phenol fumarate of the formula I comprising the characteristic band in the Raman spectrum, expressed in wave numbers (cm⁻¹), at 171 (m).

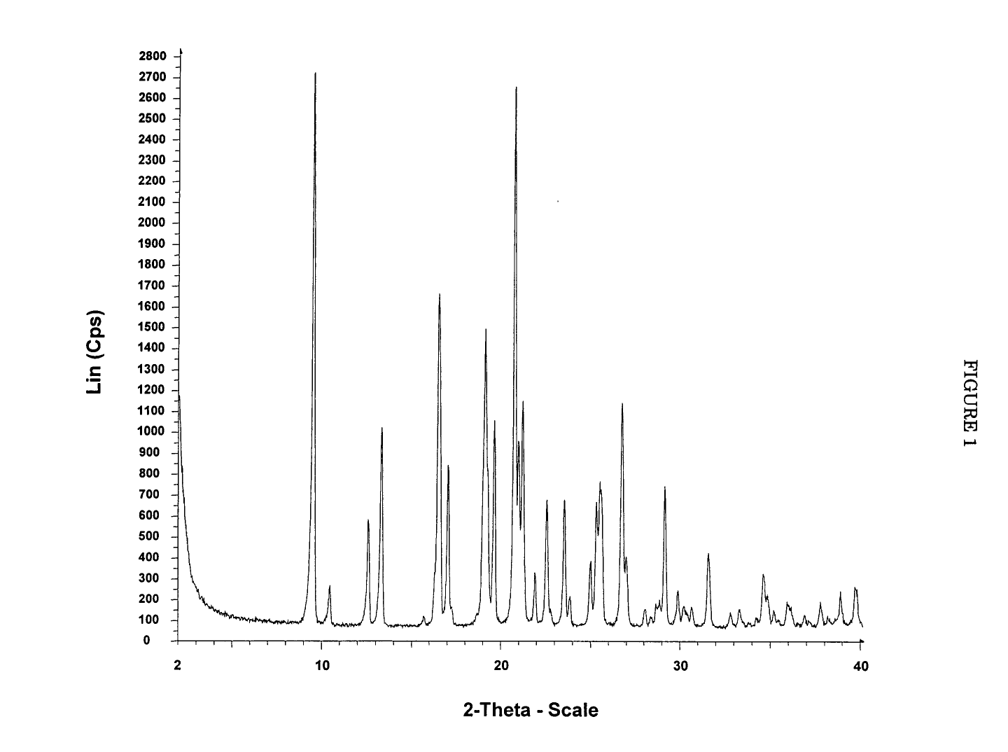

The invention also provides a crystalline form B of 3-[2-(dimethylamino)methyl-(cyclohex-1-yl)]-phenol fumarate of the formula I which has an X-ray diffraction diagram as shown in FIG. 1.

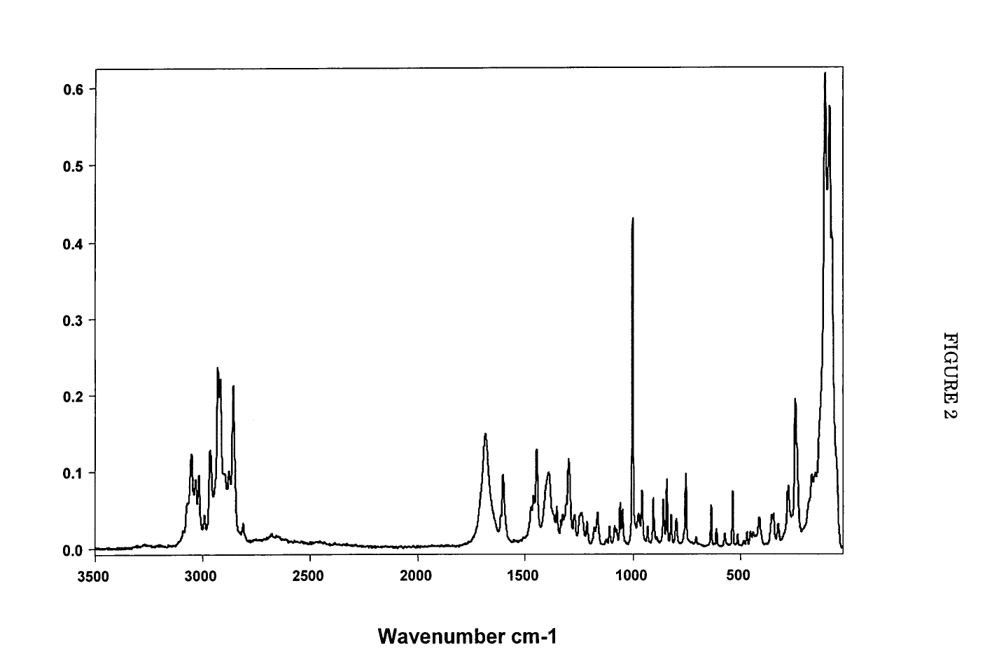

The invention also provides a crystalline form B of 3-[2-(dimethylamino)methyl-(cyclohex-1-yl)]-phenol fumarate of the formula I which has Raman spectrum as shown in FIG. 2.

The crystalline form B is the most thermodynamically stable form at low temperature up to, for example, about 40° C. and additionally has an excellent chemical and physical stability. Polymorph B is even insensitive and stable towards the influence of atmospheric humidity at high relative atmospheric humidities of up to 90%, even over a relatively long period of time. No water uptake, no formation of hydrates and no conversion into other crystalline forms are observed under normal conditions. Polymorph B also shows no phase transformations in air and in the presence of moisture. However, polymorph B changes under increased pressure or on grinding, and a conversion into the crystalline form A is observed under the action of a higher pressure. Polymorph B is not hygroscopic and absorbs only small amounts of surface water. Polymorph B also forms no solvates under these conditions, and no conversion is observed in contact with solvents. The solubility in polar solvents is very good. The melting point is about 176° C. and the melt enthalpy is about 113 J/g, determined by means of DSC at a heating up rate of 10° C./minute. The conversion temperature into the crystalline form A is, for example, above 40° C. Polymorph B can be prepared as a solid powder with the desired average particle sizes, which as a rule are in the range of from 1 μm to about 500 μm. Because of its properties, the crystalline form B is most suitable for the preparation of pharmaceutical formulations.

The compound of the formula I forms a further crystalline form B′ which is very similar to the crystalline form B, can be prepared reproducibly and, for example, in the X-ray diffraction diagram substantially differs by the intensity of the peaks and the position at 19 to 19.4° 2θ. The properties substantially correspond to form B.

The compound of the formula I forms a further crystalline form A which is thermodynamically stable at higher temperatures and is likewise stable under normal conditions in air and with exclusion of atmospheric humidity. The crystalline form A can also be handled such that it can be employed for the preparation of pharmaceutical compositions.

The invention also provides a crystalline form A of 3-[2-(dimethylamino)methyl-(cyclohex-1-yl)]-phenol fumarate of the formula I which has a characteristic X-ray diffraction diagram with the pronounced reflections given below.

| | rel. |
|---|---|
| 2 theta | intensity |
| 9.51 | 100 |
| 10.43 | 10 |
| 12.58 | 21 |
| 13.30 | 37 |
| 15.66 | 4 |
| 16.51 | 61 |
| 17.03 | 31 |
| 17.25 | 6 |
| 19.12 | 53 |
| 19.26 | 30 |
| 19.63 | 39 |
| 20.77 | 96 |
| 20.99 | 35 |
| 21.22 | 42 |
| 21.92 | 12 |
| 22.57 | 25 |
| 22.77 | 6 |
| 23.55 | 25 |
| 23.86 | 8 |
| 25.00 | 13 |
| 25.33 | 24 |
| 25.54 | 27 |
| 25.60 | 27 |
| 26.76 | 42 |
| 26.99 | 15 |
| 28.07 | 6 |
| 28.42 | 4 |
| 28.68 | 6 |
| 28.88 | 7 |
| 29.17 | 27 |
| 29.91 | 9 |
| 30.25 | 6 |
| 30.68 | 6 |
| 31.61 | 15 |
| 32.85 | 5 |
| 33.34 | 5 |
| 34.65 | 12 |
| 34.87 | 7 |
| 35.24 | 5 |
| 35.97 | 7 |
| 37.82 | 7 |
| 38.94 | 8 |
| 39.76 | 9 |
| 40.72 | 4 |
| 41.55 | 10 |
| 42.35 | 7 |
| 43.35 | 5 |
| 44.50 | 6 |
| 45.84 | 6 |
| 46.64 | 4 |

The above Table shows the peak positions (in 2 theta) and the relative intensities of the peaks, the most intense peak being standardized to a relative intensity of 100.

The invention also provides a crystalline form of 3-[2-(dimethylamino)methyl-(cyclohex-1-yl)]-phenol fumarate of the formula I which has a characteristic X-ray diffraction diagram in the range of from 2° to 35° 2θ with pronounced characteristic lines, expressed in d values (Å):
9.3 (vs), 7.0 (m), 6.7 (s), 5.36 (vs), 5.20 (m), 4.64 (vs), 4.53 (s), 4.26 (vs), 4.18 (s), 3.95 (m), 3.78 (m), 3.57 (m), 3.50 (s), 3.46 (m), 3.33 (s), 3.05 (m), 2.83 (m);
   called form A in the following.

The invention also provides a crystalline form of 3-[2-(dimethylamino)methyl-(cyclohex-1-yl)]-phenol fumarate of the formula I which has a characteristic Raman spectrum with characteristic lines, expressed in wave numbers (cm⁻¹):

| Position (cm⁻¹) | Intensity |
|---|---|
| 3070 | m |
| 3050 | m |
| 3031 | m |
| 3017 | m |
| 2991 | w |
| 2964 | m |
| 2931 | s |
| 2919 | s |
| 2898 | sh, m |
| 2872 | sh, m |
| 2859 | s |
| 2812 | vw |
| 1705 | sh, m |
| 1685 | m |
| 1644 | w |
| 1602 | m |
| 1470 | w |
| 1459 | m |
| 1445 | m |
| 1386 | m |
| 1351 | w |
| 1331 | w |
| 1323 | w |
| 1316 | w |
| 1306 | w |
| 1293 | m |
| 1269 | w |
| 1247 | w |
| 1240 | w |
| 1236 | w |
| 1211 | w |
| 1176 | w |
| 1163 | w |
| 1121 | vw |
| 1107 | w |
| 1083 | w |
| 1075 | w |
| 1058 | w |
| 1048 | w |
| 1000 | vs |
| 972 | w |
| 957 | m |
| 931 | w |
| 904 | w |
| 857 | w |
| 842 | m |
| 821 | w |
| 797 | w |
| 763 | w |
| 753 | m |
| 706 | vw |
| 634 | w |
| 610 | vw |
| 620 | vw |
| 571 | vw |
| 535 | m |
| 512 | vw |
| 468 | vw |
| 453 | vw |
| 413 | w |
| 355 | w |
| 347 | w |
| 326 | w |
| 280 | m |
| 246 | s |
| 239 | sh, m |
| 108 | vs |
| 89 | vs |
| 78 | vs |

called form A in the following.

The invention also provides a crystalline form A of 3-[2-(dimethylamino)methyl-(cyclohex-1-yl)]-phenol fumarate of the formula I comprising one or both of the characteristic bands in the Raman spectrum, expressed in wave numbers (cm⁻¹), at 1644 (w) and 1705 (sh, m).

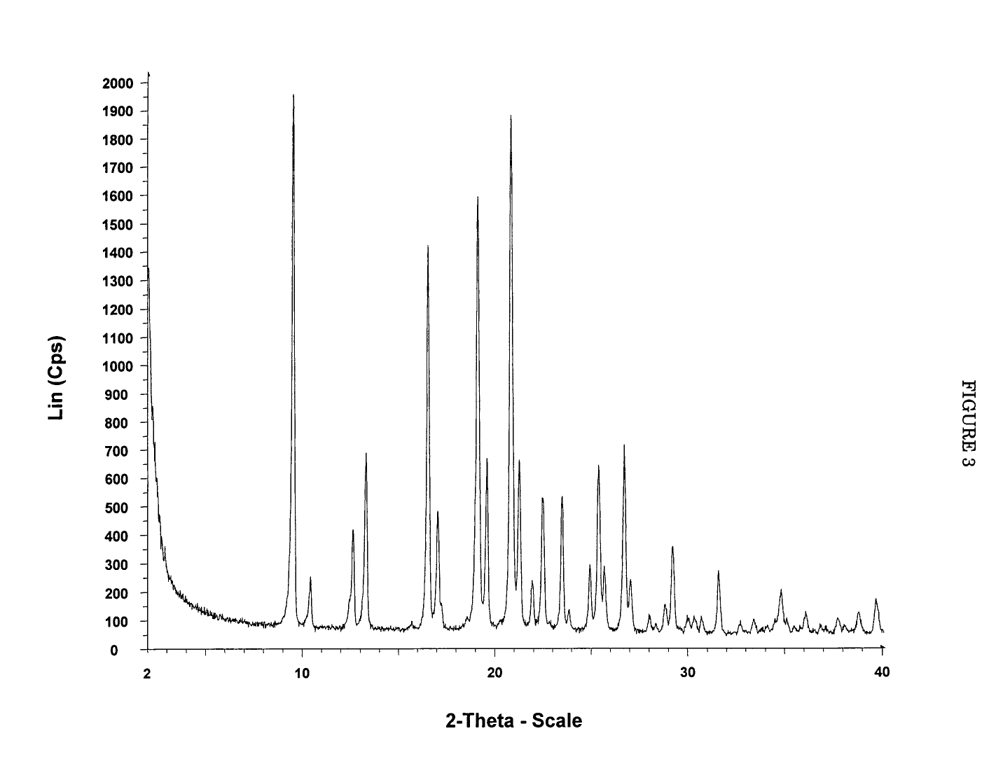

The invention also provides a crystalline form A of 3-[2-(dimethylamino)methyl-(cyclohex-1-yl)]-phenol fumarate of the formula I which has an X-ray diffraction diagram as shown in FIG. 3.

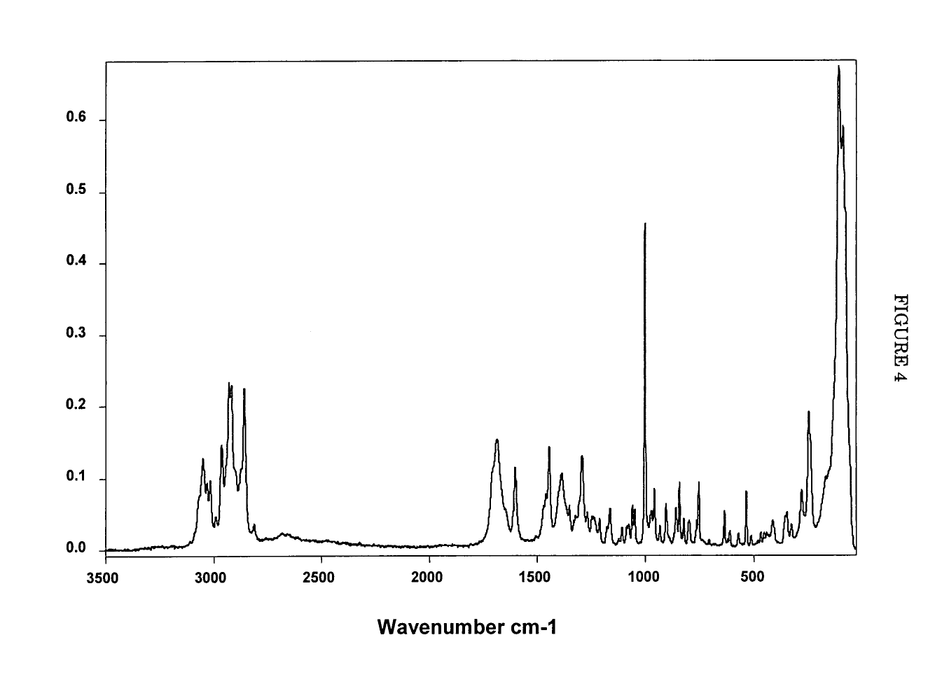

The invention also provides a crystalline form A of 3-[2-(dimethylamino)methyl-(cyclohex-1-yl)]-phenol fumarate of the formula I which has a Raman spectrum as shown in FIG. 4.

The crystalline form A is the most thermodynamically stable form at a higher temperature, for example about at least 40° C. or more. Polymorph A additionally has an excellent chemical stability at elevated temperatures also in the presence of atmospheric humidity. The physical stability is low, since a conversion into polymorph B occurs under increased pressure, and already at room temperatures, with or without the influence of moisture. However, no water uptake and no formation of hydrates are observed. Polymorph A also forms no solvates. Conversion into the crystalline form B may occur in contact with solvents. The solubility in polar solvents is very good and comparable to the solubility of polymorph B. The melting point is about 175.5° C. and the melt enthalpy is about 111 J/g, determined by means of DSC at a heating up rate of 10° C./minute. Polymorph A can be prepared as a solid powder with the desired average particle sizes, which as a rule are in the range of from 1 μm to about 500 μm. Storage of the crystalline form A under dry inert gas (for example nitrogen) is advisable.

The polymorphic form A can convert into the crystalline form B (B′) and vice versa. The forms A and B form an enantiotropic system with a transition point of about 40 to 60° C. The present invention therefore also provides mixtures of the crystalline forms A and B in any desired ratios of amounts per se. The crystal lattice of forms A and B are very similar, which manifests itself, for example, in that the Raman spectra and X-ray diffraction diagrams have only small differences. In addition to different intensities of peaks in the same position, form A has two peaks in the region of 21° 2θ and form B three peaks.

In respect of the melting points and melt enthalpies stated, it is noted that very similar values for the various crystal forms are found in differential scanning calorimetry (DSC). On temperature-dependent recording of X-ray diffraction diagrams, starting from form B, it was found that as the temperature increases, a complete conversion into the crystalline form A initially takes place (about 50° C.), and at about 150° C. a conversion into a new form C which is stable only at high temperatures and up to the melting point is then observed. It therefore cannot be ruled out that the values for the melting point and melt enthalpy are to be assigned to form C.

The polymorphic forms can be prepared by crystallization processes known per se from the salt 3-[2-(dimethylamino)methyl-(cyclohex-1-yl)]-phenol fumarate, for example stirring of suspensions (establishing of phase equilibria), precipitation, recrystallization or evaporation of solvents. Dilute, saturated or supersaturated solution can be used, with or without seeding with a crystal nucleating agent. The temperatures for the formation of solutions can be up to 100° C. The crystallization can be initiated by cooling to about −100° C. to 30° C., and preferably −30° C. to 20° C., it being possible for cooling to be carried out continuously or stepwise. For the preparation of solutions or suspensions, amorphous or crystalline starting materials can be used in order to achieve high concentrations in solutions and to obtain other crystalline forms.

The invention also provides a process for the preparation of the crystalline form B of 3-[2-(dimethylamino)methyl-(cyclohex-1-yl)]-phenol, which comprises
a) treating a pulverulent, solid, amorphous form of 3-[2-(dimethylamino)methyl-(cyclohex-1-yl)]-phenol fumarate with an inert gas containing water vapor until formation of the crystalline form B is complete; or
b) preparing a suspension of the amorphous form of 3-[2-(dimethylamino)methyl-(cyclohex-1-yl)]-phenol fumarate in a solvent, as a vehicle, and stirring the suspension until formation of the crystalline form B is complete; or
c) dissolving 3-[2-(dimethylamino)methyl-(cyclohex-1-yl)]-phenol fumarate in a solvent and then precipitating it and optionally stirring the mixture until formation of the crystalline form B is complete;
   the temperature in process stages a), b) and c) being not more than 90° C., preferably not more than 60° C., and particularly preferably not more than 40° C.

The temperature in process steps a) and b) is preferably not more than 40° C., and the process is particularly preferably carried out at room temperature. Inert gases are, for example, air, nitrogen and noble gases, air being particularly preferred merely for economic reasons. The relative humidity of the gases can be, for example, 40 to 90%, and preferably 60 to 90%. The treatment time in process stage a) substantially depends on the particle size and the relative humidity, and can be, for example, 5 to 100 hours. In process stage a), polymorph B can also be formed in addition to form A if the relative atmospheric humidity is too low and/or the treatment time is too short. After the isolation, the crystalline residue can be dried in the conventional manner, temperatures above 40° C. expediently being avoided.

Process step b) is preferably carried out at about −20° to 40° C., and particularly preferably at −5° C. to 25° C. (about room temperature). Possible solvents have been mentioned above. The treatment time in process stage b) can be 5 to 100 hours. After the isolation, the solvent or solvent mixture used can be removed in the conventional manner by means of known drying processes.

In process step c), the crystalline form A, B or the amorphous form of 3-[2-(dimethylamino)methyl-(cyclohex-1-yl)]-phenol fumarate can be used for the preparation of solutions. The concentration of 3-[2-(dimethylamino)methyl-(cyclohex-1-yl)]-phenol fumarate in the solution depends on the temperature chosen and on the solvent. The amount dissolved can be, for example, from 0.5 to 50, preferably 0.5 to 30, more preferably 0.5 to 20, and particularly preferably 1 to 15 per cent by weight, based on the solvent. The temperature for the dissolving can be up to 70° C., and preferably up to 60° C. The solution can also be prepared at room temperature if solvents having a high dissolving power are employed, for example water, acetone, dimethylformamide, dimethyl sulfoxide, methanol, ethanol, N-methylpyrrolidone and propylene glycol. The precipitation can be carried out by means of cooling, partial or complete removal of the solvent, addition of a precipitating agent (non-solvent, for example heptane or methyl t-butyl ether) or a combination of these measures. Cooling can mean slow cooling or also quenching to temperatures down to −20° C., and preferably down to 0° C. The solvent can be removed by heating, in a stream of gas, applying a vacuum or a combination of these measures. Heating to remove solvent means a temperature of not more than 40° C., and preferably not more than 30° C., in process stage c). In the precipitation processes according to process step c), the suspensions obtained are preferably subsequently stirred for a relatively long time at temperatures of −20° C., and preferably 0° C. to 10° C., in order to convert the crystalline form A possibly formed into the polymorphic form B.

The preparation of the polymorphic form B is relatively non-critical. Evaporation processes can also be employed for the preparation. In this process, attention is to be paid to the choice of the solvent and in particular the evaporation temperature. At temperatures of about room temperature, the polymorphic form B is predominantly formed. At temperatures of above 40° C., and preferably from 50° C., the polymorphic form A is predominantly formed. Furthermore, the formation of mixtures of the crystalline forms A and B is also observed at room temperature.

The same processes as for the preparation of the crystalline form B can in principle be used for targeted preparation of the crystalline form A, the temperature in the individual process steps being kept above 40° C. The temperature can be, for example, 50° C. to 120° C., and preferably 50° C. to 100° C. After the cooling, lower temperatures are preferably maintained for not more then 2, and more advantageously 1 day.

The invention also provides a process for the preparation of the crystalline form A of 3-[2-(dimethylamino)methyl-(cyclohex-1-yl)]-phenol fumarate of formula 1, which comprises
a) treating a pulverulent, solid, amorphous form of 3-[2-(dimethylamino)methyl-(cyclohex-1-yl)]-phenol fumarate with an inert gas containing water vapor until formation of the crystalline form A is complete; or
b) preparing a suspension of the amorphous form of 3-[2-(dimethylamino)methyl-(cyclohex-1-yl)]-phenol fumarate in a solvent, as a vehicle, and stirring the suspension until formation of the crystalline form A is complete; or
c) dissolving 3-[2-(dimethylamino)methyl-(cyclohex-1-yl)]-phenol fumarate in a solvent and then precipitating it and optionally stirring the mixture until formation of the crystalline form B is complete;
   the temperature in process stages a), b) and c) being more than 90° C., preferably more than 60° C., and particularly preferably more than 40° C. and the crystalline form being isolated after cooling.

On the basis of its favourable overall properties profile, 3-[2-(dimethylamino)methyl-(cyclohex-1-yl)]-phenol fumarate is outstandingly suitable as an active compound for pharmaceutical compositions, and very particularly for painkilling medicaments. The present invention accordingly also provides the use of 3-[2-(dimethylamino)methyl-(cyclohex-1-yl)]-phenol fumarate of the formula I as an active compound in medicaments, preferably as an active compound in painkillers. Diastereomers or mixtures of enantiomeric diastereomers having the trans configuration of the phenyl ring and of the dimethylaminomethyl group (1R,2R or 1S,2S configuration) are also preferred here, as in the entire Application, the enantiomer having the absolute configuration (1R,2R) being very particularly preferred.

The invention also provides a pharmaceutical composition comprising an active amount of 3-[2-(dimethylamino)methyl-(cyclohex-1-yl)]-phenol fumarate of the formula I and a pharmaceutical excipient or a pharmaceutical diluent.

The compound of the formula I can be in the composition as the crystalline form A, the crystalline form B or a mixture of forms A and B. The composition preferably comprises the crystalline form B.

The amount of compounds of the formula I substantially depends on the type of formulation and on the desired dosage during the administration period. The amount of the particular salts according to the invention to be administered to patients can vary and depends, for example, on the weight or age of the patient and on the mode of administration, the indication and the severity of the disease. 0.005 to 5,000 mg/kg, preferably 0.05 to 500 mg/kg of patient's body weight of at least one such compound are conventionally administered.

Oral formulations can be solid formulations, for example tablets, capsules, pills and lozenges. Oral formulations can also be liquid formulations, for example solutions, suspensions, syrups or elixirs. Liquid and solid formulations also include incorporation of the compounds of the formula I into solid or liquid foodstuffs. Liquids furthermore also include solutions for parenteral administration, such as, for example, infusion or injection.

The compounds of the formula I and the crystalline forms can be used directly as powders (micronized particles), granules, suspensions or solutions, or they can be mixed with other pharmaceutically acceptable ingredients and components and the mixture can then be powdered, in order for the powders then to be filled into capsules of hard or soft gelatin, to be pressed to tablets, pills or lozenges, or for the powders to be suspended or dissolved in a vehicle for the preparation of suspensions, syrups or elixirs. Tablets, pills or lozenges can be provided with a coating after pressing.

Pharmaceutically acceptable ingredients and components for the various types of formulation are known per se. They can be, for example, binders, such as synthetic or natural polymers, pharmaceutical excipients, lubricants, surfactants, sweeteners and flavorings, coating agents, preservatives, colorants, thickeners, auxiliary substances, antimicrobial agents and excipients for the various types of formulation.

Examples of binders are gum arabic, gum tragacanth, gum acacia and biodegradable polymers, such as homo- or copolyesters of dicarboxylic acids, alkylene diols, polyalkylene glycols and/or aliphatic hydroxycarboxylic acids; homo- or copolyamides of dicarboxylic acids, alkylenediamines and/or aliphatic aminocarboxylic acids; corresponding polyester/polyamide copolymers, polyanhydrides, polyorthoesters, polyphosphazenes and polycarbonates. The biodegradable polymers can be linear, branched or crosslinked. Specific examples are polyglycolic acid, polylactic acid and poly-d,l-lactic/glycolic acid. Other examples of polymers are water-soluble polymers, such as, for example, polyoxaalkylenes (polyoxaethylene, polyoxapropylene and copolymers thereof), polyacrylamides and hydroxyalkylated polyacrylamides, polyfumaric acid and esters or amides thereof, polyacrylic acid and esters or amides thereof, polyvinyl alcohol and esters or ethers thereof, polyvinylimidazole, polyvinylpyrrolidone and natural polymers, such as, for example, chitosan.

Examples of pharmaceutical excipients are phosphates, such as dicalcium phosphate.

Examples of lubricants are natural or synthetic oils, fats or waxes, or fatty acid salts, such as magnesium stearate.

Surfactants (surface-active agents) can be anionic, cationic, amphoteric or neutral. Examples of surfactants are lecithin, phospholipids, octyl sulfate, decyl sulfate, dodecyl sulfate, tetradecyl sulfate, hexadecyl sulfate and octadecyl sulfate, sodium oleate or sodium caprate, 1-acylaminoethane-2-sulfonic acids, such as 1-octanoylaminoethane-2-sulfonic acid, 1-decanoylaminoethane-2-sulfonic acid, 1-dodecanoylaminoethane-2-sulfonic acid, 1-tetradecanoylaminoethane-2-sulfonic acid, 1-hexadecanoylaminoethane-2-sulfonic acid and 1-octadecanoylaminoethane-2-sulfonic acid, bile acids, salts and derivatives thereof, such as, for example, cholic acid, deoxycholic acid, taurocholic acid, taurodeoxycholic acid and sodium glycocholates, sodium caprate, sodium laurate, sodium oleate, sodium laurylsulfate, sodium cetylsulfate, sulfated castor oil, sodium dioctylsulfosuccinate, cocamidopropylbetaine and laurylbetaine, fatty alcohols, cholesterols, glycerol mono- or distearate, glycerol mono- or dioleate, glycerol mono- or dipalmitate and polyoxyethylene stearate.

Examples of sweeteners are sucrose, fructose, lactose or aspartame.

Examples of flavorings are peppermint, wintergreen oil or fruit flavoring, such as cherry or orange flavoring.

Examples of coating agents are gelatin, waxes, shellac, sugars or biodegradable polymers.

Examples of preservatives are methyl- or propylparaben, sorbic acid, chlorobutanol and phenol.

Examples of auxiliary substances are fragrances.

Examples of thickeners are synthetic polymers, fatty acids, fatty acid salts, fatty acid esters and fatty alcohols.

Examples of liquid excipients are water, alcohols (ethanol, glycerol, propylene glycol, liquid polyethylene glycols), polytriazines and oils. Examples of solid excipients are talc, aluminas, microcrystalline cellulose, silicon dioxide, aluminum oxide and similar solids.

The composition according to the invention can also comprise isotonic agents, such as, for example, sugars, physiological buffers and sodium chloride.

The composition according to the invention can also be formulated as an effervescent tablet or effervescent powder which disintegrates in an aqueous environment with the preparation of solutions or suspensions for drinking.

A syrup or elixir can comprise the compound of the formula I, a sugar, such as sucrose or fructose, as a sweetener, a preservative (methylparaben), a colorant and a flavoring.

The composition according to the invention can also be formulations with delayed and controlled release of the active compound in contact with body fluids of the gastrointestinal tract, in order to achieve a substantially constant and effective level of the active compound in the blood plasma. For this purpose, the compounds of the formula I can be embedded into a polymer matrix of a biodegradable polymer, a water-soluble polymer or both polymers, optionally together with a suitable surfactant. In this connection, embedding can mean incorporation of microparticles into the polymer matrix. Formulations with delayed and controlled release of the active compound can also be obtained by means of encapsulation of dispersed microparticles or emulsified microdrops via known coating technologies of dispersions and emulsions.

The compounds of the formula I can also be used together with at least one further pharmaceutical active compound for combination therapies. For this, at least one further active compound can be additionally dispersed or dissolved in the composition according to the invention.

The invention also provides the use of 3-[2-(dimethylamino)methyl-(cyclohex-1-yl)]-phenol fumarate of the formula I for the preparation of a pharmaceutical composition, in particular for the treatment of states of pain.

The invention also provides a method for the treatment of states of pain, in which an active amount of 3-[2-(dimethylamino)methyl-(cyclohex-1-yl)]-phenol fumarate of the formula I is administered to a patient suffering from pain.

The medicament according to the invention (the pharmaceutical composition according to the invention) is preferably suitable for the prophylaxis and/or treatment of pain, preferably chosen from the group consisting of acute pain, chronic pain, neuropathic pain and visceral pain; of migraine; depressions; neurodegenerative diseases, preferably chosen from the group consisting of Parkinson's disease, Alzheimer's disease, Huntington's disease and multiple sclerosis; cognitive diseases, preferably cognitive deficiency states, particularly preferably attention deficit syndrome (ADS); panic attacks; epilepsy; coughing; urinary incontinence; diarrhea; pruritus; schizophrenia; cerebral ischemias; muscle spasms; spasms; food intake disorders, preferably chosen from the group consisting of bulimia, cachexia, anorexia and obesity; alcohol and/or drug (in particular nicotine and/or cocaine) and/or medicament abuse; alcohol and/or drug (in particular nicotine and/or cocaine) and/or medicament dependency, preferably for the prophylaxis and/or reduction of withdrawal symptoms with alcohol and/or drug (in particular nicotine and/or cocaine) and/or medicament dependency; development of tolerance symptoms towards medicaments; in particular towards opioids; gastro-esophageal reflux syndrome; for diuresis; for antinatriuresis; for influencing the cardiovascular system; for anxiolysis; for increasing vigilance; for increasing libido, for modulation of motor activity and for local anesthesia.

The medicament according to the invention (the pharmaceutical composition according to the invention) is particularly preferably suitable for the prophylaxis and/or treatment of pain, preferably acute pain, chronic pain, neuropathic pain or visceral pain; depressions; epilepsy; Parkinson's disease; alcohol and/or drug (in particular nicotine and/or cocaine) and/or medicament abuse; alcohol and/or drug (in particular nicotine and/or cocaine) and/or medicament dependency; preferably for the prophylaxis and/or reduction of withdrawal symptoms with alcohol and/or drug (in particular nicotine and/or cocaine) and/or medicament dependency; development of tolerance symptoms towards medicaments, in particular towards opioids, or for anxiolysis.

The medicament according to the invention (the pharmaceutical composition according to the invention) is very particularly preferably suitable for the prophylaxis and/or treatment of pain, preferably acute pain, chronic pain, neuropathic pain or visceral pain.

The use of at least one salt according to the invention, in each case optionally in the form of one of its pure stereoisomers, in particular enantiomers or diastereomers, its racemates or in the form of a mixture of stereoisomers, in particular the enantiomers and/or diastereomers, in any desired mixture ratio, and optionally one or more pharmaceutically acceptable auxiliary substances, for the preparation of a medicament for the prophylaxis and/or treatment of pain, preferably chosen from the group consisting of acute pain, chronic pain, neuropathic pain and visceral pain, of migraine, depressions, neurodegenerative diseases, preferably chosen from the group consisting of Parkinson's disease, Alzheimer's disease, Huntington's disease and multiple sclerosis, cognitive diseases, preferably cognitive deficiency states, particularly preferably attention deficit syndrome (ADS), panic attacks, epilepsy, coughing, urinary incontinence, diarrhea, pruritus, schizophrenia, cerebral ischemias, muscle spasms, spasms, food intake disorders, preferably chosen from the group consisting of bulimia, cachexia, anorexia and obesity, alcohol and/or drug (in particular nicotine and/or cocaine) and/or medicament abuse, alcohol and/or drug (in particular nicotine and/or cocaine) and/or medicament dependency, preferably for the prophylaxis and/or reduction of withdrawal symptoms with alcohol and/or drug (in particular nicotine and/or cocaine) and/or medicament dependency, development of tolerance symptoms towards drugs and/or medicaments, in particular towards opioids, gastro-esophageal reflux syndrome, for diuresis, for antinatriuresis, for influencing the cardiovascular system, for anxiolysis, for increasing vigilance, for increasing libido, for modulation of motor activity and for local anesthesia, is particularly preferred.

The medicament according to the invention can be in a liquid, semi-solid or solid pharmaceutical form, for example in the form of injection solutions, drops, juices, syrups, sprays, suspensions, tablets, patches, capsules, plasters, suppositories, ointments, creams, lotions, gels, emulsions, aerosols or in multiparticulate form, for example in the form of pellets or granules, optionally pressed to tablets, filled in capsules or suspended in a liquid, and can also be administered as such.

In addition to at least one salt according to the invention, optionally in the form of its pure stereoisomers, in particular enantiomers or diastereomers, its racemates or in the form of mixtures of the stereoisomers, in particular the enantiomers or diastereomers, in any desired mixture ratio, the medicament according to the invention conventionally comprises further physiologically acceptable pharmaceutical auxiliary substances, which can preferably be chosen from the group consisting of excipients, fillers, solvents, diluents, surface-active substances, colorants, preservatives, disintegrating agents, slip agents, lubricants, flavorings and binders.

The choice of the physiologically acceptable auxiliary substances and the amounts thereof to be employed depends on whether the medicament is to be administered orally, subcutaneously, parenterally, intravenously, intraperitoneally, intradermally, intramuscularly, intranasally, buccally, rectally or locally, for example on infections on the skin, the mucous membranes and on the eyes. Formulations in the form of tablets, coated tablets, capsules, granules, pellets, drops, juices and syrups are preferably suitable for oral administration, and solutions, suspensions, easily reconstitutable dry formulations and sprays are suitable for parenteral, topical and inhalatory administration.

Depot formulations in dissolved form or in a plaster, optionally with the addition of agents which promote penetration through the skin, are also suitable formulations for percutaneous administration.

Formulation forms which can be used orally or percutaneously can release the particular salts according to the invention in a delayed manner.

The medicaments according to the invention are prepared by means of conventional means, devices, methods and processes which are well-known from the prior art, such as are described, for example, in “Remington's Pharmaceutical Sciences”, editor A. R. Gennaro, 17th edition, Mack Publishing Company, Easton, Pa., 1985, in particular in Part 8, Chapters 76 to 93. The corresponding description is introduced herewith as reference and forms part of the disclosure.

The amount of the particular salts according to the invention to be administered to patients can vary and depends, for example, on the weight or age of the patient and on the mode of administration, the indication and the severity of the disease. 0.005 to 5,000 mg/kg, preferably 0.05 to 500 mg/kg of patient's body weight of at least one such compound are conventionally administered.

### EXAMPLES

Certain embodiments of the present invention may be further understood by reference to the following specific examples. The examples and the terminology used herein are for the purpose of describing particular embodiments only and are not intended to be limiting.

In all the DSC measurements (unless stated otherwise), the heating up rates are 10° C./minute; the temperatures stated are peak maxima.

### A) Preparation of (+)-(1R,2R)-3-[2-(dimethylamino)methyl-(cyclohex-1-yl)]-phenol fumarate

### Example A1

### Preparation as the Crystalline form B

0.0694 g (+)-(1R,2R)-3-[2-(dimethylamino)methyl-(cyclohex-1-yl)]-phenol are dissolved in 0.50 ml ethyl acetate. In addition, a second solution of 0.0356 g fumaric acid in 20 ml ethyl acetate is prepared. The second solution is added dropwise to the first solution at 40° C. and the solutions are mixed, while stirring, a white precipitate forming immediately. The mixture is cooled to 23° C., while stirring, a tacky residue forming. The residue is heated to 40° C. and then cooled to 5° C. several times, until a white solid remains. The precipitate is then filtered off over a glass frit and the residue is dried by sucking air through for 2 minutes. 0.0810 mg (78% of theory) (+)-(1R,2R)-3-[2-(dimethylamino)methyl-(cyclohex-1-yl)]-phenol fumarate is obtained as a white, crystalline solid having a melting point of about 176° C. and a melt enthalpy of about 113 J/g, determined by means of differential scanning calorimetry (DSC) at a heating up rate of 10° C./minute. The crystalline solid is the polymorphic form B, the X-ray diffraction diagram of which is shown in FIG. 1. The Raman spectrum is shown in FIG. 2.

If twice the amount of (+)-(1R,2R)-3-[2-(dimethylamino)methyl-(cyclohex-1-yl)]-phenol is employed, likewise only the 1:1 fumarate is formed.

### Example A2

### Preparation as the Crystalline Form B

0.81 g (7.0 mmol) fumaric acid are dissolved in 400 ml ethyl acetate and the solution is added dropwise to a solution of 1.64 g (7 mmol) (+)-(1R,2R)-3-[2-(dimethylamino)methyl-(cyclohex-1-yl)]-phenol in 10 ml ethyl acetate. Clouding occurs at the point of the dropwise addition. However, no solid precipitate forms. An oil initially settles out, which crystallizes only slowly. The oil is scraped off from the wall of the glass flask and the oily suspension is kept at 50 to 60° C. on a rotary evaporator, crystallization proceeding. On cooling in a refrigerator, a white, greasy precipitate settles out. This mixture is kept at 50° C. again on the rotary evaporator and then stored in a refrigerator for 3 days. The suspension is then heated to 50° C. again on the rotary evaporator and then cooled to room temperature. The now crystalline precipitate is filtered off, washed with ethyl acetate and then dried in a stream of air. The yield of rod-shaped crystals is 95.3% of theory. DSC analysis shows an endothermic signal (peak) at 175.6° C. After drying in vacuo (100 mbar) at 50° C., the following properties result: DSC peaks at 156.6 and 174.5° C., X-ray diffraction diagram corresponds to form B.

### Example A3

### Preparation as the Crystalline Form B

8.92 g fumaric acid are suspended in 120 ml ethyl acetate and the suspension is heated to 55 to 60° C. 17.96 g (+)-(1R,2R)-3-[2-(dimethylamino)methyl-(cyclohex-1-yl)]-phenol are dissolved in 108 ml ethyl acetate at 60° C. and the solution is slowly added dropwise to the suspension. A solid forms immediately at the point of the dropwise addition. When the addition of the solution is complete, the suspension is further stirred at 60° C. and a speed of rotation of the stirrer of 700 rpm for another 4 hours. It is then heated at 70° C. for a further hour and thereafter the suspension is allowed to cool to room temperature, while stirring. The crystalline solid is filtered off, washed with ethyl acetate, suspended again in ethyl acetate and then filtered off and dried in air. The yield is 26.69 g (99.3% of theory). The X-ray diffraction diagram corresponds to form B. The DSC analysis gives an endothermic signal (peak) at 175.1° C.

### Example A4

### Preparation of 3-[2-(dimethylamino)methyl-(cyclohex-1-yl)]-phenol fumarate

a) Preparation

23.16 g 3-[2-(dimethylamino)methyl-(cyclohex-1-yl)]-phenol (purity according to HPLC analysis 75.9%) are dissolved in 588 ml ethyl acetate in a 1 l flask and the solution is heated to 55° C. 8.75 g fumaric acid are than added all at once, while stirring. Agglomerates of fumaric acid which are formed are broken down with a spatula. The mixture is then stirred at 55° C. for 4 hours and, after cooling, at 20° C. for a further 2 hours. Thereafter, the crystalline precipitate is filtered off over a filter frit, washed once with 20 ml ethyl acetate and dried by sucking air through. In the thermoanalysis (DSC), a broad endothermic signal is found at 153.8° C. The purity (based on the phenol) is 89.4% according to HPLC analysis. The yield is 18.4 g (70.0% of theory).

b) Purification by Means of Washing by Stirring

The crude product prepared according to A4 a) is weighed into glass flasks, solvent is added and the mixture is treated with ultrasound (100% sound frequency) at 40° C. for 90 minutes. It is then stored at 4° C. for one hour and the solid formed is then filtered off with suction. The air-dried residues are weighed for determination of the yield and the purity is determined by HPLC analysis. The results are to be found in the following Table 1. The following abbreviations are used: EtAc is ethyl acetate, EtOH is ethanol, PrOH is i-propanol, AcNi is acetonitrile, Tol is toluene, Hex is n-hexane, DiEt is diethyl ether, BuMe is t-butyl methyl ether, Ace is acetone, MeEt is methyl ethyl ketone, THF is tetrahydrofuran.

TABLE 1

| Amount of | | | | |
|---|---|---|---|---|
| fumarate | | | | |
| (mg) | Solvent 1 (ml) | Solvent 2 (ml) | Yield (%) | Purity (%) |
| 100.6 | EtAc (2.0) | — | 95.9 | 90.0 |
| 100.6 | PrOH (2.0) | — | 66.2 | 94.9 |
| 100.7 | Hex (2.0) | — | 87.6 | 89.8 |
| 102.2 | AcNi (2.0) | — | 96.0 | 90.5 |
| 100.9 | Tol (2.0) | — | 99.5 | 89.5 |
| 100.3 | DiEt (2.0) | — | 73.9 | 89.9 |
| 101.1 | BuMe (2.0) | — | 92.4 | 89.9 |
| 101.4 | Ace (2.0) | — | 84.8 | 91.6 |
| 101.3 | MeEt (2.0) | — | 90.1 | 91.2 |
| 100.7 | THF (2.0) | — | 73.7 | 94.2 |
| 101.4 | Ace (1.98) | EtOH (0.02) | 76.2 | 92.8 |
| 101.0 | Ace 1.96) | EtOH (0.04) | 77.3 | 92.8 |
| 102.7 | Ace (1.94) | EtOH (0.06) | 72.2 | 94.1 |
| 101.7 | Ace (1.92) | EtOH (0.08) | 75.1 | 93.0 |
| 102.1 | Ace (1.90) | EtOH (0.10) | 74.2 | 93.4 |

c) Purification by Means of Washing by Stirring

The crude product obtained according to A4 a) is weighed into glass flasks, solvent is added and the mixture is shaken in a thermomixer at 50° C. for 6 hours. The solutions are then stored at 23° C. overnight and filtered, and the residue is washed with 2.5 ml of the particular solvent and then dried in air. The yield and the purity (HPLC) of the residue are determined. Further information is to be found in the following Table 2.

TABLE 2

| Amount of | | | | |
|---|---|---|---|---|
| fumarate | | | | |
| (mg) | Solvent 1 (ml) | Solvent 2 (ml) | Yield (%) | Purity (%) |
| 517.1 | PrOH (10.0) | — | 59.8 | 97.8 |
| 516.4 | Ace (10.0) | — | 73.2 | 92.7 |
| 506.9 | THF (10.0) | — | 67.6 | 96.6 |
| 518.3 | Ace (9.70) | EtOH (0.30) | 69.1 | 96.4 |

d) Purification by Means of Washing by Stirring

The crude product obtained according to A4 a) is weighed into glass flasks, solvent is added and the mixture is shaken in a thermomixer at 50° C. for 6 hours. The solutions are then stored at 4° C. overnight and filtered, and the residue is washed with 2.5 ml of the particular solvent and then dried in air. The yield and the purity (HPLC) of the residue are determined. Further information is to be found in the following Table 3.

TABLE 3

| Amount of | | | | |
|---|---|---|---|---|
| fumarate | | | | |
| (mg) | Solvent 1 (ml) | Solvent 2 (ml) | Yield (%) | Purity (%) |
| 516.5 | PrOH 10.0) | — | 62.8 | 97.4 |
| 511.2 | Ace (10.0) | — | 79.3 | 91.9 |
| 506.9 | THF (10.0) | — | 67.9 | 96.5 |
| 514.3 | Ace (9.70) | EtOH (0.30) | 68.4 | 96.3 |

e) Purification by Means of Washing by Stirring

The crude product obtained in this way is weighed into glass flasks, solvent is added and the mixture is shaken in a thermomixer at 50° C. for 6 hours. The samples are then cooled by about 5° C. in intervals of about 17 minutes down to 20° C. and the samples are then stored at 4° C. for 3 days and filtered, and the residue is washed with 2.5 ml of the particular solvent and then dried in air. The yield and the purity (HPLC) of the residue are determined. Further information is to be found in the following Table 4.

TABLE 4

| Amount of | | | | |
|---|---|---|---|---|
| fumarate | | | | |
| (mg) | Solvent 1 (ml) | Solvent 2 (ml) | Yield (%) | Purity (%) |
| 517.1 | PrOH (10.0) | — | 62.8 | 97.8 |
| 516.4 | Ace (10.0) | — | 79.3 | 91.6 |
| 506.9 | THF (10.0) | — | 67.5 | 96.5 |
| 518.3 | Ace (9.70) | EtOH (0.30) | 68.7 | 96.4 |

f) Purification by Recrystallization

500 mg of the crude product obtained according to A4 a) are introduced into a glass flask and 16 ml of a solvent mixture of 80 vol. % Ace and 20 vol. % PrOH are then added. The mixture is heated to the reflux temperature on a rotary evaporator, a further 30 ml of the solvent mixture are added and the mixture is kept on the rotary evaporator until a clear solution has formed. The solution is slowly cooled to 23° C. overnight and the crystalline precipitate is filtered off and dried in air. 308.8 mg (61.8%) of crystalline product are obtained in a purity of 97.3%.

260 mg of the crystalline product are suspended again in 20 ml of a solvent mixture of 80 vol. % Ace and 20 vol. % PrOH and the suspension is then heated under reflux for 3 hours. It is then cooled slowly to 23° C. The crystalline precipitate is filtered off and dried in air. 210.8 mg (42.2%) of crystalline product are obtained in a purity of 99.05%.

g) Purification by Recrystallization

500 mg of the crude product obtained according to A4 a) are introduced into a glass flask and 30 ml of a solvent mixture of 60 vol. % Ace and 40 vol. % PrOH are added. The mixture is heated under reflux until a clear solution is formed, the heating is switched off and the solution is then allowed to cool to room temperature overnight. The crystalline precipitate is filtered off and dried in air. 197.9 mg (39.6%) of crystalline product are obtained in a purity of 97.7%.

### Example A5

### Preparation of 3-[2-(dimethylamino)methyl-(cyclohex-1-yl)]-phenol fumarate

3-[2-(Dimethylamino)methyl-(cyclohex-1-yl)]-phenol (called base in the header of Table 5) and fumaric acid are initially introduced into a glass flask and the solvent or solvent mixture is then added. The mixture is then shaken in a heated shaker at 55° C. for 6 hours. It is then cooled down to 30° C. in the course of one hour and left to stand at room temperature overnight. The crystalline precipitate is filtered off and then dried in air. The air-dried residues are weighed for determination of the yield and the purity is determined by HPLC analysis. The results are to be found in the following Table 5. EtAc is ethyl acetate. The best purities are observed on addition of half an equivalent of fumaric acid (89.7 mg), although at only low yields.

TABLE 5

| Fumaric | | | | | |
|---|---|---|---|---|---|
| acid (mg) | Base (mg) | EtAc (ml) | PrOH (ml) | Yield (%) | Purity (%) |
| 134.7 | 360.4 | 5.57 | 0.62 | 56.83 | 85.84 |
| 179.9 | 360.2 | 5.40 | 1.35 | 79.91 | 89.08 |
| 179.7 | 360.3 | 6.75 | — | 57.54 | 81.35 |
| 89.9 | 360.2 | 5.63 | — | 42.04 | 79.76 |
| 89.7 | 360.4 | 5.63 | — | 48.83 | 81.05 |
| 135.4 | 360.1 | 5.57 | 0.62 | 44.43 | 92.33 |
| 89.6 | 360.0 | 4.50 | 1.12 | 38.47 | 92.35 |
| 89.9 | 360.1 | 4.50 | 1.13 | 39.36 | 92.92 |
| 179.4 | 360.3 | 6.75 | — | 64.10 | 81.37 |
| 179.4 | 360.5 | 5.40 | 1.35 | 90.49 | 83.80 |
| 134.7 | 360.0 | 5.57 | 0.62 | 47.10 | 80.76 |

### B) Preparation of amorphous (+)-(1R,2R)-3-[2-(dimethylamino)methyl-(cyclohex-1-yl)]-phenol fumarate

### Example B1

### Freeze Drying

601.2 mg of the fumarate prepared according to Example A1 are dissolved in 8 ml water and the solution is then quenched to −85° C. It is then freeze-dried at this temperature and under a pressure of 0.250 mbar for 21 hours. A solid, white residue, which is still amorphous in the presence of 22% relative atmospheric humidity and at 25° C. even after 2 months, is obtained in a quantitative yield. A glass transition temperature close to 55° C. is determined by means of differential scanning calorimetry (DSC, heating rate 10° C./minute). An exothermic peak at 118° C. is attributed to crystallization. The crystalline material formed melts at about 175.5° C., which indicates the formation of the polymorphic form A.

### Example B2

### Freeze Drying

500.7 mg of the fumarate prepared according to Example A1 are dissolved in 8 ml water and the solution is then quenched to −80° C. It is then freeze-dried at this temperature and under a pressure of 0.570 mbar for 20 hours. A solid, white residue, which is amorphous, is obtained in a quantitative yield.

### C) Preparation of the Crystalline Form A

Unless stated otherwise, the crystal forms are determined by comparison of the Raman spectra.

### Example C1

### Phase Equilibria with Suspensions in Ethyl Acetate

25 ml ethyl acetate are added to 101 mg of the fumarate prepared according to Example Al and the mixture is heated to 70° C., while stirring. It is then stirred at this temperature for 2 hours. Thereafter, it is cooled to room temperature and the solid is filtered off and dried in a stream of air at room temperature. 87 mg of the crystalline form A are obtained as a white powder.

### Example C2

### Crystallization from Ethyl Acetate

80 mg of the fumarate prepared according to Example A1 are dissolved in 60 ml ethyl acetate at 65° C. The clear solution obtained is first cooled to room temperature and kept at this temperature for 2 hours. A white precipitate forms. The suspension is left to stand at 5° C. for a further 18 hours. The precipitate is then filtered off and the residue is dried in a stream of air at room temperature. 55 mg of the crystalline form A are obtained in the form of a white powder. The crystalline solid has a melting point of 175.5° C. and a melt enthalpy of 111 J/g, determined by means of differential scanning calorimetry (DSC) at a heating up rate of 10° C./minute. The crystalline solid is the polymorphic form A, the X-ray diffraction diagram of which is shown in FIG. 3. The Raman spectrum is shown in FIG. 4.

### Example C3

### Crystallization from Ethyl Acetate

400 mg of the fumarate prepared according to Example A1 are dissolved in 450 ml ethyl acetate at 65° C. in the course of 2 hours. The clear solution obtained is first cooled to room temperature and kept at this temperature for 2.5 hours, no precipitate forming. The solution is left to stand at 5° C. for a further 4 days. The white precipitate formed is then filtered off and the residue is dried in a stream of air at room temperature. 301 mg of the crystalline form A are obtained in the form of a white powder.

### Example C4

### Crystallization from Tetrahydrofuran (THF)

50 mg of the fumarate prepared according to Example A1 are dissolved in 4 ml THF at 60° C. in the course of 45 minutes. The solution is then cooled with dry ice, no precipitate forming. The solution is left to stand at −18° C. for a further 18 days. The white precipitate formed is then filtered off and the residue is dried in a stream of air at room temperature. 12 mg of the crystalline form A are obtained in the form of a white powder.

### Example C5

### Crystallization from Isopropanol by Means of Evaporation

29 mg of the amorphous fumarate prepared according to example B2 are dissolved in 6 ml isopropanol at room temperature and the solution is filtered through a 0.22 μm filter. Thereafter, the solution is stirred in an open vessel at 50° C. for 2 days. It is then cooled to room temperature and the remaining solvent is evaporated in a stream of nitrogen. The solid residue is the crystalline form A.

### D) Preparation of the crystalline form B

### Example D1

80.1 mg of the amorphous fumarate prepared according to Example B1 are suspended in 2 ml acetone and the suspension is stirred at 23° C. for 24 hours. It is then filtered and the residue is dried in a stream of air at room temperature. 50 mg of the crystalline form B are obtained in the form of a white powder. The melting point, melt enthalpy, X-ray diffraction diagram and Raman spectrum correspond to the values, diagram and spectrum given in Example A1.

### Example D2

80 mg of the amorphous fumarate prepared according to Example B1 are suspended in 2 ml dioxane and the suspension is stirred at 23° C. for 24 hours. It is then filtered and the residue is dried in a stream of air at room temperature. 35 mg of the crystalline form B are obtained in the form of a white powder.

### Example D3

80 mg of the amorphous fumarate prepared according to Example B1 are suspended in 2 ml ethyl acetate and the suspension is stirred at 23° C. for 24 hours. It is then filtered and the residue is dried in a stream of air at room temperature. 50 mg of the crystalline form B are obtained in the form of a white powder.

### Example D4

80 mg of the amorphous fumarate prepared according to Example B1 are suspended in 2.5 ml isopropanol and the suspension is stirred at 23° C. for 24 hours. It is then filtered and the residue is dried in a stream of air at room temperature. 36 mg of the crystalline form B are obtained in the form of a white powder.

### Example D5

80 mg of the amorphous fumarate prepared according to Example B1 are suspended in 2 ml tetrahydrofuran and the suspension is stirred at 23° C. for 24 hours. It is then filtered and the residue is dried in a stream of air at room temperature. 44 mg of the crystalline form B are obtained in the form of a white powder.

### Example D6

80 mg of the amorphous fumarate prepared according to Example B1 are suspended in 2 ml tert-butyl methyl ether and the suspension is stirred at 23° C. for 24 hours. It is then filtered and the residue is dried in a stream of air at room temperature. 58 mg of the crystalline form B are obtained in the form of a white powder.

### Example D7

40 mg of the amorphous fumarate prepared according to Example B1 are suspended in 0.02 ml water and the suspension is stirred at 23° C. for 24 hours. It is then filtered and the residue is dried in a stream of air at room temperature. 18 mg of the crystalline form B are obtained in the form of a white powder.

### Example D8

25 mg of the fumarate prepared according to Example A1 and 25 mg of the fumarate prepared according to Example C3 are suspended in 1 ml isopropanol and the suspension is stirred at 23° C. for 24 hours. It is then filtered and the residue is dried in a stream of air at room temperature. 31 mg of the crystalline form B are obtained in the form of a white powder.

### Example D9

24 mg of the fumarate prepared according to Example D2 and 20 mg of the fumarate prepared according to Example A1 are suspended in 5 ml isopropanol and the suspension is stirred at 23° C. for 19 hours. It is then filtered and the residue is dried in a stream of air at room temperature. 3.3 mg of the crystalline form B are obtained in the form of a white powder.

### Example D10

20 mg of the amorphous fumarate prepared according to Example B2 are suspended in 2 ml ethyl acetate and the suspension is stirred at 23° C. for 1 month. It is then filtered and the residue is dried in a stream of air at room temperature. 12 mg of the crystalline form B are obtained in the form of a white powder.

### Example D11

20 mg of the amorphous fumarate prepared according to Example B2 are suspended in 2 ml ethyl acetate and the suspension is stirred at 40° C. for 1 month. It is then filtered and the residue is dried in a stream of air at room temperature. 11 mg of the crystalline form B are obtained in the form of a white powder.

### Example D12

20 mg of the amorphous fumarate prepared according to Example B2 are suspended in 2 ml toluene and the suspension is stirred at 40° C. for 1 month. It is then filtered and the residue is dried in a stream of air at room temperature. 6 mg of the crystalline form B are obtained in the form of a white powder.

### Example D13

### Recrystallization from Isopropanol

2.0 g of the fumarate salt prepared according to Example A4a are suspended in 30 ml isopropanol in a stirred flask and the suspension is heated to 80° C. in the course of 10 minutes at a speed of rotation of the stirrer of about 600 rpm (revolutions per minute), a clear solution being formed. The speed of rotation is increased to 800 rpm and the solution is stirred at this temperature for 10 minutes, the speed of rotation being increased briefly to 1,000 rpm towards the end. The temperature is then lowered to 10° C. in the course of 155 minutes with an increasing cooling rate. The white solid is filtered off and dried in air (1.315 g, 65.8%). According to the X-ray powder diffractogram, only lines of the crystalline form B are measured. The thermoanalysis (DSC) shows endothermic signals at 152.4° C. and 174.0° C. No significant weight loss is observed in the thermogravimetric analysis, so that no solvated form can be present. The purity is 97.9%, determined by HPLC.

### Example D14

### Recrystallization from Isopropanol

2.0 g of the fumarate salt prepared according to Example A4a are suspended in 30 ml isopropanol in a stirred flask and the suspension is heated to 80° C. in the course of 10 minutes at a speed of rotation of the stirrer of about 600 rpm (revolutions per minute), a clear solution being formed. The speed of rotation is increased to 800 rpm and the solution is stirred at this temperature for 10 minutes, the speed of rotation being increased briefly to 1,000 rpm towards the end. The temperature is then lowered to 10° C. in the course of 140 minutes with a linear cooling rate of 0.2 K/min. The white solid is filtered off and dried in air (1.318 g, 65.9%). According to the X-ray powder diffractogram, only lines of the crystalline form B are measured. The thermoanalysis (DSC) shows endothermic signals at 151.9° C. and 174.2° C. No significant weight loss is observed in the thermogravimetric analysis, so that no solvated form can be present. The purity is 98.0%, determined by HPLC.

### E) Stability Investigations

### Example E1

### Storage of the Amorphous Form in High and Low Atmospheric Humidity

a) 30 mg of the amorphous fumarate prepared according to Example Bi are stored at room temperature and 75% relative atmospheric humidity for 5 days. Thereafter, the amorphous product has converted completely into the crystalline form B.

b) 30 mg of the amorphous fumarate prepared according to Example B2 are stored at room temperature and 90% relative atmospheric humidity for 3 days. Thereafter, the amorphous product has converted completely into the crystalline form B.

c) 30 mg of the amorphous fumarate prepared according to Example B2 are stored at room temperature and 53% relative atmospheric humidity for 2 months. Thereafter, the amorphous product has converted completely into the crystalline form B.

d) 30 mg of the amorphous fumarate prepared according to Example B2 are stored at room temperature and 22% relative atmospheric humidity for 2 months. Thereafter, the amorphous product is still present.

### Example E2

### Storage of the Crystalline form A in High Atmospheric Humidity

The fumarate prepared according to Example C3 is stored at room temperature and 75% relative atmospheric humidity, and after 3 days and 1, 2, 3, 4 and 8 weeks samples are analyzed by means of Raman spectroscopy. The crystalline form A has already converted into the crystalline form B after 3 days, and after 8 weeks the crystalline form B is also present.

### Example E3

### Storage of the Crystalline Form B in High Atmospheric Humidity

The crystalline form B prepared according to Example A1 is stored at room temperature and 75% relative atmospheric humidity, and after 3 days and 1, 2, 3, 4 and 8 weeks samples are analyzed by means of Raman spectroscopy. The samples are practically unchanged even after 8 weeks.

### Example E4

### Stability Under Grinding Conditions

a) Crystalline form A on Trituration

The fumarate prepared according to Example C3 is triturated with a pestle in a mortar dish for 5 minutes. Thereafter, a mixture of the crystalline form A and B is present.

b) Crystalline form B on Trituration

The crystalline form B according to Example A1 is triturated with a pestle in a mortar dish for 5 minutes. Thereafter, the crystalline form B is present in unchanged form.

c) Crystalline form A on Grinding

The fumarate prepared according to Example A1 is introduced into an agate ball mill (Retsch MM200 mixing mill with 5 mm agate balls) and ground at 20 Hz and room temperature for 180 minutes. Thereafter, a mixture of the crystalline form A and B is present.

d) Crystalline form B on Grinding

The procedure according to Example E4c) is followed with the crystalline form B according to Example A1. After 180 minutes a mixture of the crystalline forms A and B is present.

### Example E5

### Stability Under Pressure

a) Crystalline Form A During Pressing of Tablets

The fumarate prepared according to Example C3 is introduced into a tablet press and pressed to a tablet under a pressure of 100 MPa in vacuo for 60 minutes. Thereafter, a mixture of the crystalline forms A and B is present.

b) Crystalline Form B During Pressing of Tablets

The procedure according to Example E5a) is followed with the crystalline form B according to Example A1. After 60 minutes a mixture of the crystalline forms A and B is present.

### Example E6

### Water Uptake

The water uptake is determined by means of dynamic water vapor uptake (dynamic vapor sorption, DVS) with the DVS-1 apparatus from Surface Measurement Systems Ltd. The sample is placed in a platinum crucible on the tip of a microbalance. The sample is then first equilibrated at 50% relative atmospheric humidity and subsequently subjected to a predefined measurement program. The temperature is 25° C. The change in weight of the sample is determined.

a) Amorphous Form

The amorphous form has a pronounced water uptake of about 6.5 wt. % up to about 77% relative atmospheric humidity. At a higher atmospheric humidity the weight decreases again, which is caused by crystallization. After the end of the experiment, the crystalline form A is present.

b) Crystalline Form A

The crystalline form A has a water uptake of only about 0.2 wt. %. At the end of the measurement cycle, substantially the crystalline form B is present.

c) Crystalline Form B

The crystalline form B has a water uptake of only about 0.2 wt. %. At the end of the measurement cycle, the crystalline form B is present in unchanged form.

### Example E7

### Stability at Elevated Temperature

The crystalline forms are stored in open and closed vessels and possible changes are measured as stability parameters by chromatography (HPLC) after a given time. The crystalline form A proves to be very stable in respect of chemical stability and crystal form. The crystalline form B proves to be very stable in respect of chemical stability. The results are given in the following Table 6.

TABLE 6

| Polymorph | Conditions | Time | HPLC | FT Raman¹⁾ |
|---|---|---|---|---|
| B | 40° C./75% r.h. | 1 week | 100.0% | B |
| B | 60° C./75% r.h. | 4 weeks | 99.1% | B |
| B | 60° C. (closed) | 1 week | 99.0% | B |
| B | 60° C. (closed) | 4 weeks | 100.6% | B |
| B | −18° C. (reference) | 4 weeks | 100% | B |
| A | 40° C./75% r.h. | 1 week | 99.5% | B |
| A | 60° C./75% r.h. | 4 weeks | 99.3% | B |
| A | 60° C. (closed) | 1 week | 99.9% | A and B |
| A | 60° C. (closed) | 4 weeks | 99.6% | A and B |
| A | −18° C. (reference) | 4 weeks | 100% | A and B |

¹⁾Determination of the crystalline form

### Example E8

### Water Uptake of Fumarate and Hydrochloride (Comparison)

The water uptake is determined by means of dynamic water vapor uptake (dynamic vapor sorption, DVS) with the DVS-1 apparatus from Surface Measurement Systems Ltd. The sample is placed in a platinum crucible on the tip of a microbalance. The sample is then first equilibrated at 50% relative atmospheric humidity and subsequently subjected to a predefined measurement program. The temperature is 25° C. The change in weight of the sample after a stepwise increase in the relative atmospheric humidity by in each case 10% to 90% is determined.

### a) (+)-(1R,2R)-3-[2-(Dimethylamino)methyl-(cyclohex-1-yl)]-phenol fumarate (form B)

A decidedly low water uptake of 0.13% is found. The moisture absorbed is released again completely at about 10% relative atmospheric humidity.

### b) (+)-(1R,2R)-3-[2-(Dimethylamino)methyl-(cyclohex-1-yl)]-phenol hydrochloride (form D)

On increasing the relative atmospheric humidity to about 75%, a water uptake of about 2 wt. % is observed. A further increase to 90% relative atmospheric humidity leads to a water uptake of about 5 wt. % in total. On desorption by stepwise lowering of the relative atmospheric humidity by 10%, more water is released than is taken up before, namely 8.5% in total. It is assumed that a variable hydrate is present.

### Example E9

### Damp Storage and Drying of Fumarate and Hydrochloride (Comparison)

Fumarate and hydrochloride according to Example E8 are first stored at 25° C. and 75% and 95% relative atmospheric humidity for 7 days and thereafter at 50° C. in vacuo in a drying cabinet for 7 days. Before the start of the storage, the X-ray diffraction diagrams (XRD), DSC for determination of transition points (Tp) and dry weight (DW) and the Karl Fischer water content (WC) are determined, and the same values are determined after the damp storage and drying. The X-ray diffraction diagrams show no change in the crystalline forms A and D after damp storage and drying. The starting values for the hydrochloride are Tp=116.27° C., DW=6.35% and WC=8.00%. The starting values for the fumarate are Tp=93.11° C. and 175.14° C., DW=0 and WC=0.2%. The results are given in Tables 7 (75% relative atmospheric humidity) and 8 (95% relative atmospheric humidity).

TABLE 7
(75%):

| | | DW | WC | | DW | WC |
|---|---|---|---|---|---|---|
| Salt | Tp (° C.) | (%) | (%) | Tp (° C.) | (%) | (%) |
| Fumarate | 91.97/175.14 | — | 0.50 | 92.67/174.83 | — | 0.30 |
| Hydrochloride | 116.22 | 5.92 | 8.50 | 113.83 | 6.12 | 6.00 |

TABLE 8
(95%):

| | | DW | WC | | DW | WC |
|---|---|---|---|---|---|---|
| Salt | Tp (° C.) | (%) | (%) | Tp (° C.) | (%) | (%) |
| Fumarate | 92.63/175.20 | — | 0.50 | 92.67/174.89 | — | 0.30 |
| Hydrochloride | 116.52 | 7.84 | 8.50 | 114.47 | 6.74 | 6.60 |

Apparatuses, methods:

Differential scanning calorimetry (DSC): Apparatus name Perkin Elmer DSC 7 or Perkin Elmer Pyris 1. Variable measurements (heating rate) in gold or aluminum crucibles.

Powder X-ray diffraction diagrams (PXRD):

PXRD is carried out with a Philips 1710 powder X-ray diffractometer, CuKₐ radiation being used. d intervals are calculated from the 2θ values, the wavelength of 1.54060 Å being taken as the basis. It generally applies that the 2θ values have an error rate of ±0.1-0.2°. The experimental error in the d interval values therefore depends on the location of the line (the peak).

Raman Spectroscopy:

FT-Raman spectra are recorded with a Bruker RFS 100 FT-Raman system which is operated with an Nd:YAG laser (wavelength 1064 nm) and a germanium detector cooled with liquid nitrogen. For each sample, 64 scans with a resolution of 2 cm⁻¹ are accumulated. A laser output of 100 mW is generally used.

The foregoing description and examples have been set forth merely to illustrate the invention and are not intending to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A salt of fumaric acid and 3-[2-(dimethylamino)methyl-(cyclohex-1-yl)]-phenol.

2. The salt of claim 1, wherein said salt is a 3-[2-(dimethylamino)methyl-(cyclohex-1-yl)]-phenol fumarate compound corresponding to formula I

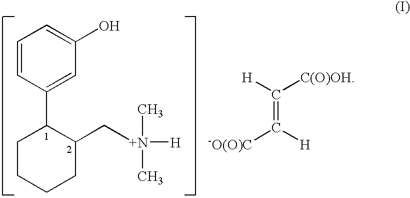

3. The salt of claim 2, wherein the 3-[2-(dimethylamino)methyl-(cyclohex-1-yl)]-phenol fumarate compound corresponding to formula I is in the form of a diastereomer or mixture of enantiomeric diastereomers having the trans configuration of the phenyl ring and of the dimethylaminomethyl group (1R,2R or 1S,2S configuration)

4. The salt of claim 2, wherein the 3-[2-(dimethylamino)methyl-(cyclohex-1-yl)]-phenol fumarate compound corresponding to formula I has the 1R,2R configuration.

5. A process for preparing a 3-[2-(dimethylamino)methyl-(cyclohex-1-yl)]-phenol fumarate compound corresponding to formula I, comprising:
combining 3-[2-(dimethylamino)methyl-(cyclohex-1-yl)]-phenol and fumaric acid to form a 3-[2-(dimethylamino)methyl-(cyclohex-1-yl)]-phenol fumarate compound corresponding to formula I

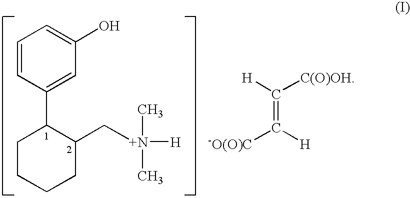

6. The process of claim 5, wherein at least one of the components is present in dissolved or suspended form.

7. The process of claim 5, comprising the steps of:
a) dissolving or suspending 3-[2-(dimethylamino)methyl-(cyclohex-1-yl)]-phenol in a solvent, or introducing 3-[2-(dimethylamino)methyl-(cyclohex-1-yl)]-phenol into a reaction vessel in undissolved form,
b) mixing the solution, suspension or undissolved 3-[2-(dimethylamino)methyl-(cyclohex-1-yl)]-phenol with fumaric acid or a suspension or a solution of fumaric acid in a solvent to form a mixture, and
c) isolating a compound corresponding to formula I.

8. The process of claim 7, further comprising cooling the mixture.

9. The process of claim 8, wherein the temperature does not exceed 90° C.

10. A salt of fumaric acid and 3-[2-(dimethylamino)methyl-(cyclohex-1-yl)]-phenol produced by the process set forth in claim 5.

11. A crystalline form B of a 3-[2-(dimethylamino)methyl-(cyclohex-1-yl)]-phenol fumarate compound corresponding to formula I of claim 2, having a characteristic X-ray diffraction diagram in the range of from 2° to 35° 2θ with the following pronounced characteristic lines, expressed in d values (Å):
9.3 (vs), 7.0 (m), 6.7 (s), 5.37 (s), 5.21 (s), 4.64 (s), 4.52 (s), 4.28 (vs), 4.23 (s), 4.19 (s), 3.94 (m), 3.78 (m), 3.52 (m), 3.49 (m), 3.33 (s), 3.30 (m), 3.06 (s), 2.83 (m).

12. The compound of claim 11, wherein the compound has a 1R,2R configuration.

13. A crystalline form B of a 3-[2-(dimethylamino)methyl-(cyclohex-1-yl)]-phenol fumarate compound corresponding to formula I of claim 2, having a characteristic X-ray diffraction diagram as shown in FIG. 1.

14. The compound of claim 13, wherein the compound has a 1R,2R configuration.

15. A crystalline form B of a 3-[2-(dimethylamino)methyl-(cyclohex-1-yl)]-phenol fumarate compound corresponding to formula I of claim 2, having a characteristic Raman spectrum comprising the following band, expressed in wave numbers (cm⁻¹): 171 (m).

16. The compound of claim 15, wherein the compound has a 1R,2R configuration.

17. A crystalline form B of a 3-[2-(dimethylamino)methyl-(cyclohex-1-yl)]-phenol fumarate compound corresponding to formula I of claim 2, having a Raman spectrum as shown in FIG. 2.

18. The compound of claim 17, wherein the compound has a 1R,2R configuration.

19. A crystalline form A of a 3-[2-(dimethylamino)methyl-(cyclohex-1-yl)]-phenol fumarate compound corresponding to formula I of claim 2, having a characteristic X-ray diffraction diagram in the range of from 2° to 35° 2θ with the following pronounced characteristic lines, expressed in d values (Å): 9.3 (vs), 7.0 (m), 6.7 (s), 5.36 (vs), 5.20 (m), 4.64 (vs), 4.53 (s), 4.26 (vs), 4.18 (s), 3.95 (m), 3.78 (m), 3.57 (m), 3.50 (s), 3.46 (m), 3.33 (s), 3.05 (m), 2.83 (m).

20. The compound of claim 19, wherein the compound has a 1R,2R configuration.

21. A crystalline form A of a 3-[2-(dimethylamino)methyl-(cyclohex-1-yl)]-phenol fumarate compound corresponding to formula I of claim 2, having a characteristic X-ray diffraction diagram as shown in FIG. 3.

22. The compound of claim 21, wherein the compound has a 1R,2R configuration.

23. A crystalline form A of a 3-[2-(dimethylamino)methyl-(cyclohex-1-yl)]-phenol fumarate compound corresponding to formula I of claim 2, having a characteristic Raman spectrum comprising one or both of the following bands, expressed in wave numbers (cm⁻¹): 1644 (w) and 1705 (sh, m).

24. The compound of claim 23, wherein the compound has a 1R,2R configuration.

25. A crystalline form A of a 3-[2-(dimethylamino)methyl-(cyclohex-1-yl)]-phenol fumarate compound corresponding to formula I of claim 2, having a Raman spectrum as shown in FIG. 4.

26. The compound of claim 25, wherein the compound has a 1R,2R configuration.

27. A process for preparing a crystalline form of 3-[2-(dimethylamino)methyl-(cyclohex-1-yl)]-phenol fumarate, comprising
treating a pulverulent, solid, amorphous form of 3-[2-(dimethylamino)methyl-(cyclohex-1-yl)]-phenol fumarate with an inert gas containing water vapor to form crystalline 3-[2-(dimethylamino)methyl-(cyclohex-1-yl)]-phenol fumarate; or
preparing a suspension of an amorphous form of 3-[2-(dimethylamino)methyl-(cyclohex-1-yl)]-phenol fumarate in a solvent, and stirring the suspension to form crystalline of 3-[2-(dimethylamino)methyl-(cyclohex-1-yl)]-phenol fumarate; or
dissolving 3-[2-(dimethylamino)methyl-(cyclohex-1-yl)]-phenol fumarate in a solvent and precipitating the resulting mixture and optionally stirring the mixture to form crystalline 3-[2-(dimethylamino)methyl-(cyclohex-1-yl)]-phenol; and
preventing the temperature from exceeding 90° C.

28. A crystalline form of 3-[2-(dimethylamino)methyl-(cyclohex-1-yl)]-phenol fumarate corresponding to formula I: 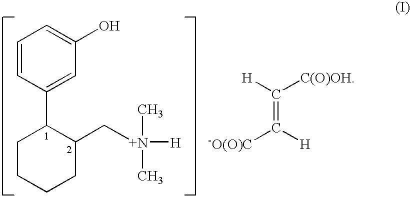
produced by the process set forth in claim 27.

29. A pharmaceutical composition comprising an physiologically active amount of the 3-[2-(dimethylamino)methyl-(cyclohex-1-yl)]-phenol fumarate compound set forth in claim 2 and a pharmaceutical excipient or a pharmaceutical diluent.

30. A composition as claimed in claim 29, wherein the compound of the formula I is in crystalline form A, crystalline form B or in a mixture of forms A and B,
wherein
crystalline form A has:
a characteristic X-ray diffraction diagram in the range of from 2° to 35° 2θ with the following pronounced characteristic lines, expressed in d values (Å):
9.3 (vs), 7.0 (m), 6.7 (s), 5.36 (vs), 5.20 (m), 4.64 (vs), 4.53 (s), 4.26 (vs), 4.18 (s), 3.95 (m), 3.78 (m), 3.57 (m), 3.50 (s), 3.46 (m), 3.33 (s), 3.05 (m), 2.83 (m), or
a characteristic X-ray diffraction diagram in the range of from 2° to 35° 2θ with the following pronounced characteristic lines, expressed in d values (Å):
9.3 (vs), 7.0 (m), 6.7 (s), 5.36 (vs), 5.20 (m), 4.64 (vs), 4.53 (s), 4.26 (vs), 4.18 (s), 3.95 (m), 3.78 (m), 3.57 (m), 3.50 (s), 3.46 (m), 3.33 (s), 3.05 (m), 2.83 (m), or
a characteristic X-ray diffraction diagram as shown in FIG. 3, or
a characteristic Raman spectrum comprising one or both of the following bands, expressed in wave numbers (cm⁻¹): 1644 (w) and 1705 (sh, m), or
a Raman spectrum as shown in FIG. 4, and
crystalline form B has:
a characteristic X-ray diffraction diagram in the range of from 2° to 35° 2θ with the following pronounced characteristic lines, expressed in d values (Å):
9.3 (vs), 7.0 (m), 6.7 (s), 5.37 (s), 5.21 (s), 4.64 (s), 4.52 (s), 4.28 (vs), 4.23 (s), 4.19 (s), 3.94 (m), 3.78 (m), 3.52 (m), 3.49 (m), 3.33 (s), 3.30 (m), 3.06 (s), 2.83 (m), or
a characteristic X-ray diffraction diagram as shown in FIG. 1, or
a characteristic Raman spectrum comprising the following band, expressed in wave numbers (cm⁻¹): 171 (m), or
a Raman spectrum as shown in FIG. 2.

31. A composition as claimed in claim 30, which comprises the compound corresponding to formula I as crystalline form B.

32. A method for treating or alleviating pain, said method comprising administering to a patient suffering from pain an effective amount of a 3-[2-(dimethylamino)methyl-(cyclohex-1-yl)]-phenol fumarate compound corresponding to formula I of claim 2.
